SEQUENCE LISTING (13 sequences)

SEQ ID NO: 1 - protein, 16 aa, Homo sapiens
RRTEEENLRKKGEPHH

SEQ ID NO: 2 - protein, 16 aa, Homo sapiens
CRTEEENLRKKGEPHH

SEQ ID NO: 3 - protein, 392 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 4 - protein, 572 aa, synthetic construct
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 5 - protein, 214 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - protein, 117 aa, synthetic construct
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSS

SEQ ID NO: 7 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIK

SEQ ID NO: 8 - protein, 5 aa, synthetic construct
SYVMH

SEQ ID NO: 9 - protein, 17 aa, synthetic construct
YINPYNDGTKYNEKFKG

SEQ ID NO: 10 - protein, 8 aa, synthetic construct
GGYYAMDY

SEQ ID NO: 11 - protein, 11 aa, synthetic construct
RASQDISNYLN

SEQ ID NO: 12 - protein, 7 aa, synthetic construct
YTSRLHS

SEQ ID NO: 13 - protein, 9 aa, synthetic construct
QQGNTLPYT

(19) United States
(12) Patent Application Publication
(10) Pub. No.: US 2024/0124565 A1
(43) Pub. Date: Apr. 18, 2024

(54) ANTIBODY BINDING A LINEAR EPITOPE OF HUMAN P53 AND DIAGNOSTIC APPLICATIONS THEREOF

(71) Applicant: Diadem SpA, Milan (IT)

(72) Inventors: Maurizio Memo, Monticelli Brusati (IT); Daniela Letizia Uberti, Brescia (IT)

(21) Appl. No.: 18/475,473

(22) Filed: Sep. 27, 2023

Related U.S. Application Data
(63) Continuation of application No. 17/528,946, filed on Nov. 17, 2021, now Pat. No. 11,802,148.
Application No. 17/528,946 is a continuation of application No. 17/093,541, filed on Nov. 9, 2020, now Pat. No. 11,208,473.
Application No. 17/093,541 is a continuation of application No. 16/214,455, filed on Dec. 10, 2018, now Pat. No. 10,875,908.
Application No. 16/214,455 is a continuation of application No. 15/515,693, filed on Mar. 30, 2017, now Pat. No. 10,183,990.

(30) Foreign Application Priority Data
Sep. 30, 2014 (IT) TO2014A000776

(51) Int. Cl. C07K 16/18; C07K 16/32; G01N 33/68

(52) U.S. Cl. CPC C07K 16/18; C07K 16/32; G01N 33/6896; C07K 2317/34; G01N 2333/4748; G01N 2800/2821; G01N 2800/56; G01N 2800/7042

(57) ABSTRACT

The invention relates to an anti-human p53 antibody suitable for specifically binding a linear epitope which is exposed only in a conformationally altered isoform of the characteristic p53 protein of patients with Alzheimer's disease or prone to develop Alzheimer's disease or cognitive impairment during ageing. Methods and diagnostic and prognostic kits are also described.

### CROSS REFERENCE TO RELATED APPLICATION

The present application is a continuation of and claims priority to U.S. patent application Ser. No. 17/528,946, filed Nov. 17, 2021, and entitled “Antibody Binding a Linear Epitope of Human p53 and Diagnostic Applications Thereof,” which is a continuation of and claims priority to U.S. patent application Ser. No. 17/093,541, filed Nov. 9, 2020, and entitled “Antibody Binding a Linear Epitope of Human p53 and Diagnostic Applications Thereof,” and which issued as U.S. Pat. No. 11,208,473 on Dec. 28, 2021, which is a continuation of and claims priority to U.S. patent application Ser. No. 16/214,455, filed Dec. 10, 2018, and entitled “Antibody Binding a Linear Epitope of Human p53 and Diagnostic Applications Thereof,” and which issued as U.S. Pat. No. 10,875,908 on Dec. 29, 2020, which is a continuation of and claims priority to U.S. patent application Ser. No. 15/515,693, filed Mar. 30, 2017, and entitled “Antibody Binding a Linear Epitope of Human p53 and Diagnostic Applications Thereof” and which issued as U.S. Pat. No. 10,183,990 on Jan. 22, 2019, which is a U.S. national stage of and claims priority to International Patent Application No. PCT/EP2015/072094, filed Sep. 25, 2015, which claims priority to and the benefit of Italian Patent Application No. TO2014A000776, filed Sep. 30, 2014, the contents of each of which are herein incorporated by reference in their entirety.

### SEQUENCE LISTING

This application contains a Sequence Listing which has been submitted electronically as a WIPO Standard ST.26 XML file via Patent Center, created on Jan. 8, 2024, is entitled “121689-10106.xml” and is 13.3 KB in size. The sequence listing is incorporated herein by reference in its entirety.

### BACKGROUND OF THE INVENTION

The present invention lies in the field of immunodiagnostics.

More particularly, the present invention relates to an antibody, a kit and in vitro methods of diagnosing Alzheimer's disease and/or the predisposition of a subject to develop Alzheimer's disease or cognitive impairment during ageing.

The DNA binding domain (DBD) of human p53 protein (aa 101-306) is characterized by high-grade conformational flexibility and contains one Zn²⁺ ion coordinated by three cysteines (residues 176, 238 and 242) and one histidine (residue 179). In the wild-type isoform of human p53 protein, the aa 282-297 linear epitope is masked and therefore not available for recognition by an epitope-specific antibody.

However, because of the high-grade flexibility of its DBD domain, p53 protein can assume several conformations, which also characterize its biological activity.

In its wild-type conformation, p53 can bind DNA consensus sequences and transcribe/repress the expression of target genes. In this conformation, the protein exposes an epitope which is recognized by the commercially available, conformationally specific antibody named PAb1620.

There are several grades of conformational alteration states of p53, which correspond to different conformationally altered isoforms. Some mutations in the p53 gene cause a conformational change of the protein, which corresponds to a conformationally altered isoform capable of being recognized by commercially available, conformationally specific antibodies.

Additional conformationally altered isoforms may also derive from post-translational modifications, such as oxidation and/or nitration reactions, which alter the wild-type tertiary structure of the protein.

Buizza L. et al. (“Conformational altered p53 as an early marker of oxidative stress in Alzheimer's disease”, PlosOne 7(1):e29789) and Uberti D et al. (“Identification of a mutant-like conformation of p53 in fibroblasts from sporadic Alzheimer's disease patients”, Neurobiology of Aging 27 (2006) 1193-1201) both identify the expression of mutated p53 protein in patients with Alzheimer's disease, such identification having been made using the anti-p53 antibody PAb240 which binds a cryptic epitope on the amino acid residues 213-217 of p53 protein, not detectable in the wild-type p53 protein.

### BRIEF SUMMARY OF THE INVENTION

The present inventors have unexpectedly found an anti-human p53 which has proved capable of specifically and selectively recognize a conformationally altered isoform of the protein resulting from its post-translational modifications, in which the aa 282-297 linear epitope is exposed.

The inventors have also unexpectedly observed that the isoform of p53 protein recognized by the antibody of the invention is peculiarly expressed in subjects with Alzheimer's disease. In particular, the conformationally altered isoform recognized by the antibody of the invention is expressed in higher amounts in biological samples—particularly in samples of blood cells, neuronal cells or other cell types, as well as in samples of biological fluids such as, for example, blood, plasma, serum, saliva, urine—of patients with Alzheimer's disease.

The inventors further observed that such conformationally altered isoform recognized by the antibody of the invention is also expressed in biological samples of subjects with Mild Cognitive Impairment (MCI).

Therefore, the antibody of the invention is a useful diagnostic and prognostic tool, for diagnosing Alzheimer's disease and for determining the predisposition of a subject with Mild Cognitive Impairment (MCI) to develop Alzheimer's disease.

Finally, the inventors observed that the expression of the p53 protein isoform positive to the antibody of the invention in a statistically significant manner correlates with the age and cognitive impairment of the subjects studied.

Therefore, the antibody of the invention is also useful for determining the predisposition of a subject to develop cognitive impairment during ageing.

### BRIEF DESCRIPTION OF THE DRAWINGS

FIG. 1 discloses a P53 protein sequence;

FIG. 2 discloses experimental data of 2d3A8/1620 ratio versus CONT, SAD and FAD;

FIG. 3 discloses experimental data of example 4 of unfolded P53 PBMC 2d3A8 absorbance versus CONT, MCI and AD;

FIG. 4 discloses experimental data of example 5, during ageing, of unfolded P53 PBMC 2d3A8 absorbance versus CONT, MCI and AD; and

FIG. 5 discloses experimental data of example 5, decreasing of the scoring in the MMSE test, of unfolded P53 PBMC 2d3A8 absorbance versus CONT, MCI and AD.

### DETAILED DESCRIPTION OF THE INVENTION

A first object of the present invention is therefore an anti-human p53 antibody, characterized in that it recognizes the linear epitope of sequence RRTEEENLRKKGEPHH (SEQ ID NO:1) present in the DNA binding domain (DBD) of human p53, said linear epitope spanning between the amino acid positions 282-297 of the amino acid sequence of human p53.

The preparation of the antibody of the invention is described in the following experimental part.

In a preferred embodiment, the antibody of the invention is a monoclonal antibody.

The antibody of the present invention can be obtained by any well-known methodology for the preparation of polyclonal or monoclonal antibodies. In the following experimental part, the preparation of the antibody by animal (mice) immunization with an antigen consisting of a peptide of sequence CRTEEENLRKKGEPHH (SEQ ID NO:2) conjugated with bovine serum albumin as carrier and hybridoma technique is described by way of example.

As previously mentioned, the antibody object of the present invention specifically recognizes an isoform of human p53 protein which has been shown to be correlated to Alzheimer's disease and development of cognitive impairment during ageing. Such antibody therefore represents a useful diagnostic and prognostic tool.

An in vitro method of determining an isoform conformationally altered by post-translational modification of p53 protein peculiar of Alzheimer's disease, as well as the diagnostic and prognostic methods as defined in the appended claims which form an integral part of the present description, are also part of the present invention.

The method detects the formation of an immunocomplex between the human p53 protein and the antibody of the sample.

An immunodiagnostic kit as defined in the appended claims is also part of the present invention.

In order to implement the methods and kit of the invention any type of well-known immunoassay can be used, such as for example immunoprecipitation assay, ELISA or RIA, immunofluorescence, Western Blot, FACS analysis, immunocytochemistry/immunohistochemistry.

An immunoassay kit may include the antibody and means for detecting the binding of the antibody to human p53 protein.

The following non-limitative examples are provided to illustrate the scope of the invention as defined by the appended claims.

### Example 1

### 1a. Immunization

For the immunization, 6/8 week old mice which were healthy and disorder-free were used. The peptide used as antigen for antibody production had the following features:
Sequence: “N-terminal” CRTEEENLRKKGEPHH “C terminal” (SEQ ID NO: 2)
Length: 16 amino acids
Molecular weight: 1960.94
Purity: 96.4%
Form: lyophilized powder
Conjugation: Conjugated with BSA by the glutaraldehyde method

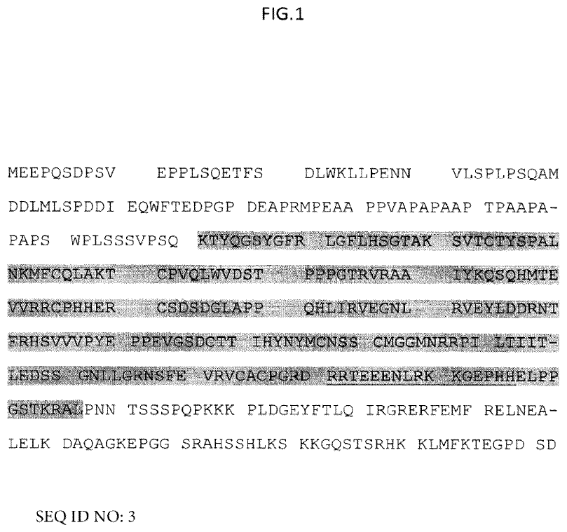

The p53 protein sequence (SEQ ID NO: 3) is depicted in FIG. 1, where the DNA binding domain is highlighted in grey, and the linear epitope recognized by 2D3A8 antibody is underlined. The amino acid sequences of the 2D3A8 antibody include the heavy chain (SEQ ID NO: 4) and light chain (SEQ ID NO: 5), heavy chain variable region (SEQ ID NO: 6) and light chain variable region (SEQ ID NO: 7), heavy chain CDRs 1, 2 and 3 (SEQ ID NOS: 8, 9 and 10, respectively) and light chain CDRs 1, 2 and 3 (SEQ ID NOS: 11, 12 and 13, respectively).

The first injection has been performed by emulsifying the antigen (50 μg) in Freund's complete adjuvant (FCA). The subcutaneous injections have been performed in 2-3 sites on the animal. Additional injections have been performed at 3-week intervals with 50 μg of antigen emulsified in Freund's incomplete adjuvant (FIA). The antibody titer is evaluated by ELISA.

In the ELISA assay, the antibody titer present in the serum of 5 mice was evaluated after the third injection with the above-described peptide. The blood of immunized mice was collected from their caudal veins. The absorbance values obtained following the spectrophotometric reading provided important information on the antibody titer present in the various mice. Animals were subjected to further additional injections so that the antibody titer reached a sufficiently high level. The mouse with the best antibody titer was chosen for the first fusion.

### 1b. Development of Hybridomas

The animals' splenocytes were fused with mouse myeloma cells (SP2/O cell line). The fusion products were subjected to screening against the antigen to select the antibody-producing clones. The growth of these clones was continued. This first screening was performed by ELISA methodology. The positive clones were labeled as “parental clones” and frozen after 3 passages. An antigen coating was created onto ELISA plates, and successively the fusion product supernatant was added. The serum of the immunized animals was used as a positive control in ELISA (Example 2).

### Example 2

After the fusion between the splenocytes of the mouse with the best antibody titer and mouse myeloma cells, an ELISA assay was performed to evaluate the fusion products. A coating with the antigen was created in a 96-well ELISA plate and serial dilutions of the supernatant from various clones were added to each well to evaluate their antibody production by spectrophotometric reading. The clones with the highest optical density at 450 nm (OD₄₅₀ₙₘ) were transferred to 24-well plates and after their growth the ELISA assay was repeated, the clones with the highest antibody production were transferred to 6-well plates, grown and tested again by ELISA. The procedure was repeated also for the clones transferred to culture flasks. These successive assays allowed the identification of the best clones which were tested for the last time by ELISA, with the limit dilution method to ensure that the positive clones showed an actual antibody response.

The validated antibody was purified from the supernatant of the clone with the highest OD_{450 nm} value and therefore with the best antibody titer. This antibody is named “clone 2D3A8” for brevity.

### Example 3

Study of the expression of the conformationally altered isoform of p53 protein recognized by 2D3A8 antibody in patients with sporadic and familial Alzheimer's disease and MCI.

In immortalized B lymphocytes of patients diagnosed with sporadic Alzheimer (SAD) and familial Alzheimer (FAD), the conformational state of p53 was evaluated by the immunoprecipitation method, using two conformationally specific antibodies which recognize the wild-type isoform of the protein (PAb 1620) and a conformationally altered isoform (2D3A8). The immunoprecipitate was then visualized by Western Blot with a polyclonal anti-p53 antibody (CM1). The experimental data were expressed as ratio between the intensity of the band positive to 2D3A8 antibody and to PAb1620 of the same sample.

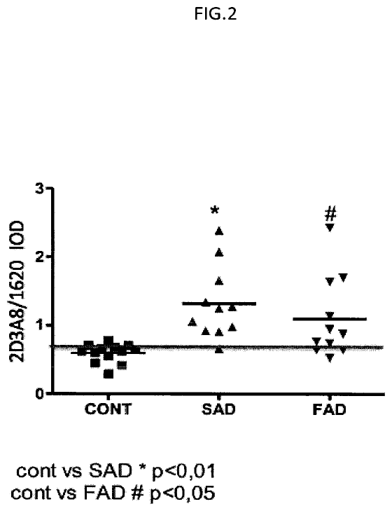

In SAD and FAD samples, the 2D3A8/1620 ratio was significantly higher compared to the lymphocytes of dementia-free control patients (FIG. 2).

2D3A8 antibody can therefore discriminate a conformationally altered isoform of p53 peculiarly expressed in immortalized lymphocytes of patients with sporadic (SAD) and familial (FAD) Alzheimer.

### Example 4

In samples of fresh blood of patients diagnosed Alzheimer and of subjects with mild cognitive impairment, diagnosed MCI, the conformationally altered isoform of p53 recognized by 2D3A8 antibody (2D3A8-positive p53) was evaluated by ELISA. Healthy, dementia-free subjects of the same age were also evaluated.

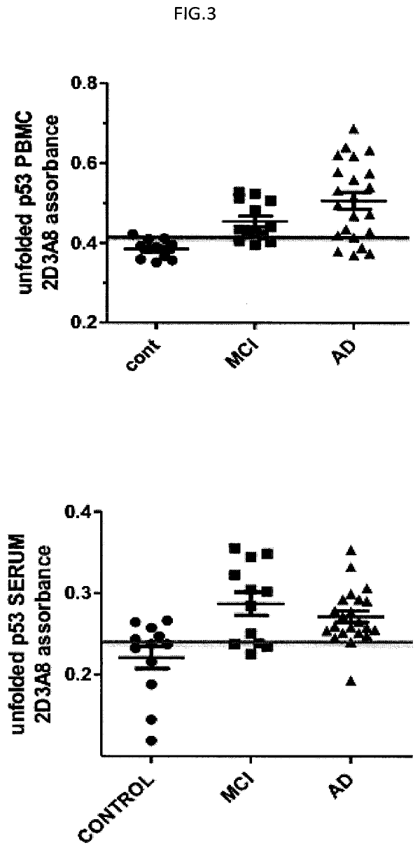

2D3A8-positive p53 vas detected both in blood cells (PBMC) and in serum of the same patients or subjects. 2D3A8 antibody can recognize with high-specificity grade patients with Alzheimer. Interestingly, subjects with mild cognitive impairment express serum levels of 2D3A8-positive p53 statistically higher than the levels of protein isoform present in control subjects. In PBMCs and in serum of patients with Alzheimer, the 2D3A8-positive p53 isoform was statistically increased compared to the controls (FIG. 3).

### Example 5

The 2D3A8-positive p53 isoform correlates with age.

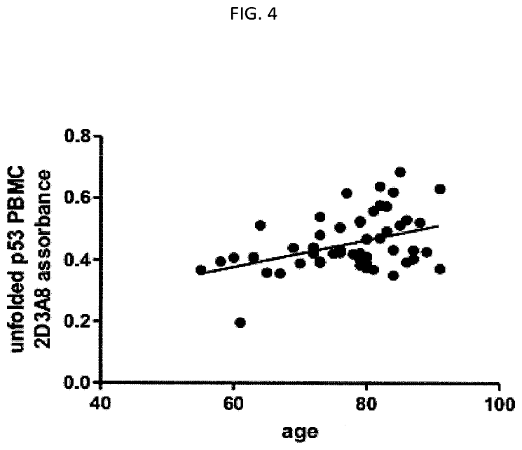

During ageing, the expression of 2D3A8-positive p53 in blood cells (PBMC) increases in a statistically significant manner (FIG. 4).

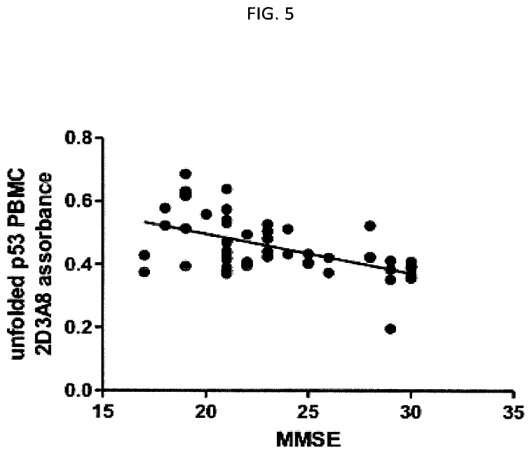

Moreover, the expression of 2D3A8-positive p53 correlates with the cognitive status, measured by the well-known neuropsychological test MMSE. The 2D3A8-positive p53 isoform increases with the decrease of the scoring obtained in the MMSE test, i.e., it increases with the progression of cognitive impairment (FIG. 5).

1. A method of providing an immune complex comprising an isoform of human p53 protein and an anti-human p53 antibody, the method comprising the steps of:
a. contacting a biological sample of a subject with an anti-human p53 antibody in a reaction mixture, wherein said biological sample comprises a human p53 isoform that is conformationally altered with respect to the wild-type human p53 protein and said antibody comprises a heavy chain variable region comprising CDR1 (SEQ ID NO:8), CDR2 (SEQ ID NO: 9) and CDR3 (SEQ ID NO: 10) and a light chain variable region comprising CDR1 (SEQ ID NO:11), CDR2 (SEQ ID NO: 12) and CDR3 (SEQ ID NO: 13), under conditions which permit formation in said reaction mixture of an immune complex between said antibody and said human p53 protein isoform, and
b. isolating from said reaction mixture said immune complex, thereby providing said immune complex.

2. The method of claim 1, further comprising the step of quantifying the amount of said altered isoform of human p53 protein in said immune complex.

3. The method of claim 1, wherein said isolating of step b. comprises immunoprecipitation.

4. The method of claim 1, wherein said heavy chain variable region comprises SEQ ID NO: 6 and said light chain variable region comprises SEQ ID NO: 7.

5. The method of claim 1, wherein the antibody comprises a heavy chain comprising SEQ ID NO:4 and a light chain comprising SEQ ID NO:5.

6. The method of claim 2, wherein said quantifying comprises using one or more of immunoprecipitation, ELISA, MA, immunofluorescence, Western Blot, FACS, immunocytochemistry and immunohistochemistry.

7. The method of claim 1, wherein said biological sample comprises lymphocytes, plasma, serum or blood.

8. A method of providing a quantified immune complex comprising an isoform of human p53 protein and an anti-human p53 antibody, the method comprising the steps of:
a. contacting a biological sample of a subject in a reaction mixture with an anti-human p53 antibody, wherein said sample comprises an human p53 isoform that is conformationally altered with respect to the wild-type human p53 protein and wherein said antibody comprises a heavy chain variable region comprising CDR1 (SEQ ID NO:8), CDR2 (SEQ ID NO: 9) and CDR3 (SEQ ID NO: 10) and a light chain variable region comprising CDR1 (SEQ ID NO:11), CDR2 (SEQ ID NO: 12) and CDR3 (SEQ ID NO: 13), under conditions which permit formation in said reaction mixture of an immune complex between said antibody and said human p53 protein isoform, and
b. quantifying the amount of said altered isoform of human p53 protein in said immune complex in said reaction mixture, thereby providing said quantified immune complex.

9. The method of claim 8, wherein said heavy chain variable region comprises SEQ ID NO: 6 and said light chain variable region comprises SEQ ID NO: 7.

10. The method of claim 8, wherein the antibody comprises a heavy chain comprising SEQ ID NO:4 and a light chain comprising SEQ ID NO:5.

11. The method of claim 8, wherein said quantifying comprises using one or more of immunoprecipitation, ELISA, RIA, immunofluorescence, Western Blot, FACS, immunocytochemistry and immunohistochemistry.

12. The method of claim 8, wherein said biological sample comprises lymphocytes, plasma, serum or blood.

13. A method of providing a detectable immune complex comprising an isoform of human p53 protein and a detectable anti-human p53 antibody, the method comprising the steps of:
a. contacting a biological sample in a reaction mixture with an anti-human p53 monoclonal antibody, wherein said sample comprises a human p53 isoform that is conformationally altered with respect to wild-type human p53 protein, wherein said antibody comprises a heavy chain variable region comprising CDR1 (SEQ ID NO:8), CDR2 (SEQ ID NO: 9) and CDR3 (SEQ ID NO: 10) and a light chain variable region comprising CDR1 (SEQ ID NO:11), CDR2 (SEQ ID NO: 12) and CDR3 (SEQ ID NO: 13), and wherein said detectable antibody permits its detection, under conditions which permit formation in said reaction mixture of an immune complex between said antibody and said human p53 protein isoform; and
b. isolating said immune complex from said reaction mixture, thereby providing said detectable immune complex.

14. The method of claim 13, wherein said heavy chain variable region comprises SEQ ID NO: 6 and said light chain variable region comprises SEQ ID NO: 7.

15. The method of claim 13, wherein the antibody comprises a heavy chain comprising SEQ ID NO:4 and a light chain comprising SEQ ID NO:5.

16. The method of claim 13, wherein said immune complex is isolated by immunoprecipitation.

17. The method of claim 16, wherein said immune complex is detectable using one or more of, ELISA, RIA, immunofluorescence, Western Blot, FACS, immunocytochemistry and immunohistochemistry.

18. The method of claim 1, wherein said biological sample comprises lymphocytes, plasma, serum or blood.
